(19) United States
(12) Patent Application Publication
(10) Pub. No.: US 2018/0073023 A1
(43) Pub. Date: Mar. 15, 2018

(54) CIRCULAR SINGLE-STRANDED NUCLEIC ACID, METHOD FOR PREPARING THE SAME, AND METHOD FOR USING THE SAME

(71) Applicant: HITACHI, LTD., Tokyo (JP)

(72) Inventors: Hiroko MATSUNAGA, Tokyo (JP); Maiko TANABE, Tokyo (JP)

(21) Appl. No.: 15/690,699

(22) Filed: Aug. 30, 2017

(30) Foreign Application Priority Data
Sep. 9, 2016 (JP) 2016-177054

(51) Int. Cl. C12N 15/113; A61K 31/70; C12Q 1/68

(52) U.S. Cl. CPC C12N 15/113; A61K 38/00; C12Q 1/6813; A61K 31/70

(57) ABSTRACT

An object of the present invention is to provide a circular single-stranded nucleic acid, and a method for preparing the same and a method for using the same. A circular single-stranded nucleic acid according to one embodiment of the present invention is a circular single-stranded nucleic acid for determining a target base on a genomic DNA, and includes a first single-stranded nucleic acid which has the target base or a complementary base thereto and is a part of one of the strands of the genomic DNA, and a second single-stranded nucleic acid which has an index sequence to serve as an index of a cell, from which the genomic DNA is derived, or a complementary sequence thereto.

### TECHNICAL FIELD

The present invention relates to a circular single-stranded nucleic acid, a method for preparing the same, and a method for using the same.

### BACKGROUND ART

In an organism, the information of a sequence on a genomic DNA is transcribed into mRNA, and a protein is synthesized based on the information. Functions of such synthesized proteins in the body of the organism maintain the biological activity. On the other hand, the recent advancement of next generation sequencer technology allows us to determine each of the genomic DNA sequences of individual cells. A movement that the information of the genomic DNAs thus obtained which has been used for basic research such as elucidation of mechanism of development is applied to medical treatments has become active. Analyzes of the genomic DNAs of individual cells have uncovered the fact that a tissue which appears to be composed uniformly of the same cell is in a non-uniform state in which a variety of cells are present in admixture. In particular, in cancer, it is considered that the non-uniformity of cancer cell population worsens the prognosis. That is, it is considered that a group of a few cell which could not be eliminated by a selected therapeutic method proliferate thereafter to cause recurrence of the cancer. One of the causes of this non-uniformity of cancer tissues is considered to lie in genomic mutations which vary among cells. The form of the genomic mutations includes a point mutation occurring in only one base and an abnormality of a large genomic region or the whole chromosome. These abnormalities are known to accumulate in a cancer with the progress of the cancer. In order to elucidate a genomic mutation which is one of the causes of non-uniformity of cancer tissue, it is essential to analyze the genomic DNA at the single cell level such that individual cells are analyzed, which is expected to allow us to elucidate the mechanism of transformation into a cancer and perform an appropriate treatment based on the finding.

In order to analyze mutations in a genome derived from a single cell or a very small number of cells such as a few to about 100 cells, first of all, it is necessary to amplify a genomic DNA in which two copies are present per cell. The following methods for amplifying the whole genomic DNA derived from a single cell, which are categorized according to the step, are known: (1) a method based on PCR, (2) a method based on a strand displacement reaction, and (3) a method combining a strand displacement reaction with PCR.

In the method based on PCR of (1), a product is obtained by allowing random primers (i.e., primers having approximately 6 to 15-mer random sequences) to bind to a genomic DNA and performing an extension reaction. The product can be obtained by repeating the following 3 steps several tens of times: a denaturation step (94 to 96° C.) of a synthesized DNA and a template DNA, an annealing step (about 50 to 60° C.) between the primer and the template DNA, and a DNA extension step (about 65 to 75° C.). There are many devised protocols in which by decreasing the temperature (annealing temperature) when annealing complementary strands, the efficiency of annealing complementary strands is increased, and thereafter, by increasing the annealing temperature, the DNA synthesis accuracy is increased. This is applied to PicoPLEX WGA kit (New England Biolabs Ltd.) or GenomePlex Single Cell Whole Genome Amplification Kit (SIGMA, Inc.). The advantage of this method is that the method does not pass through a complicated reaction step. On the other hand, this method is not suitable for amplifying the whole genomic DNA without bias because there is a remarkable difference in facility of amplification depending on the regions due to their sequences.

The MDA method of (2) is the same as (1) up to the step of allowing random primers (generally 6-mer) to complementarily bind to a genomic DNA and synthesizing a strand. However, this method is different from PCR in that an enzyme Phi29 DNA polymerase to be used in the subsequent reaction forces this synthesized strand to dissociate from the template DNA, and a next random primer becomes a site where a complementary strand is synthesized again. This method is also characterized in that both the strand displacement reaction and the strand synthesis reaction are constant temperature (30° C.) reactions unlike PCR in which a high temperature and a low temperature are repeatedly cycled. Since the reactions are constant temperature reactions, the length of the synthesized strand is very long. Further, the great characteristic of this method is that the accuracy of DNA synthesis (accuracy of base synthesis) of this method is nearly 1000 times higher than that of the standard PCR, and false-positive or negative results can be excluded. This is applied to REPLI-g Single Cell Kit (QIAGEN, Inc.) or True Prime Single Cell WGA Kit (SYGNIS, Inc.).

The MALBAC method of (3) includes a little complicated step as compared with the methods of (1) and (2). By conecting a specific sequence (adaptor sequence) to an end of random primers, a strand having adaptor sequences added to both ends forms a loop structure in the pre-amplification step. A product having a loop structure never serves as a template again in the pre-amplification step. In both of the PCR of (1) and the MDA method of (2), synthesized products dissociate from the templates by increasing the temperature or by using a strand displacement activity and converted to single strands, whereby the synthesized products are used as templates again, and this step is considered to increase the bias of sequence-specific amplification. This problem is solved in the MALBAC method by allowing the products to forma loop structure so that they never serves as templates. In the pre-amplification step, DNA is linearly amplified, and a genomic DNA in which only two copies are present is amplified in a given amount, and thereafter, it is amplified to a desired amount by PCR. This is applied to MALBAC Single Cell WGA Kit (Yikon Genomics Co., Ltd.).

All of the methods are alleged to be able to amplify the whole genomic DNA derived from a single cell, and are techniques capable of being applied similarly to the amplification of a genomic DNA derived from a very small number of cells.

### SUMMARY OF INVENTION

### Technical Problem

An object of the present invention is to provide a circular single-stranded nucleic acid, and a method for preparing the same and a method for using the same.

### Solution to Problem

An embodiment of the present invention is a circular single-stranded nucleic acid for determining a target base on a genomic DNA, and includes a first single-stranded nucleic acid which is a part of one of the strands of the genomic DNA and has the target base or a complementary base thereto, and a second single-stranded nucleic acid which has an index sequence to serve as an index of a cell, from which the genomic DNA is derived, or a complementary sequence thereto. This circular single-stranded nucleic acid may include a third single-stranded nucleic acid which has an adaptor sequence or a complementary sequence thereto adjacent to the second single-stranded nucleic acid.

Another embodiment of the present invention is a method for preparing a circular single-stranded nucleic acid for determining a target base on a genomic DNA, which includes a step of obtaining an amplification product by performing a nucleic acid amplification reaction using a first oligonucleotide and a second oligonucleotide as primers and using the genomic DNA as a template to obtain an amplification product, the first oligonucleotide including a second single-stranded nucleic acid having an index sequence to serve as an index of a cell, from which the genomic DNA is derived and a fourth single-stranded nucleic acid having a random sequence in this order from the 5′ side, a second oligonucleotide including a fifth single-stranded nucleic acid having a nucleotide sequence residing 1 to 1000 bases apart from the target base or a complementary sequence thereto, and a step of connecting both ends of one of the single-stranded nucleic acids of the amplification product to circularize the single-stranded nucleic acid. In this method, the first oligonucleotide may include a third single-stranded nucleic acid having an adaptor sequence at the 5′ side of the second single-stranded nucleic acid. Further, the fourth single-stranded nucleic acid may be a population of single-stranded nucleic acids having a random sequences. The first oligonucleotide may be bound to a solid-phase substrate.

A still another embodiment of the present invention is a method for preparing a nucleic acid for determining a target base on a genomic DNA, which includes a step of performing a nucleic acid amplification reaction using a first oligonucleotide and a second oligonucleotide as primers and using the genomic DNA as a template to obtain an amplification product, a second single-stranded nucleic acid having an index sequence to serve as an index of a cell, from which the genomic DNA is derived and a fourth single-stranded nucleic acid having a random sequence in this order from the 5′ side, a second oligonucleotide including a fifth single-stranded nucleic acid having a first neighboring nucleotide sequence residing 1 to 1000 bases apart from the target base or a complementary base thereto; a step of connecting both ends of one of the single-stranded nucleic acids of the amplification product to circularize the nucleic acid to obtain a circular single-stranded nucleic acid; and a step of performing a rolling circle amplification (RCA) reaction using the circular single-stranded nucleic acid as a template and using a third oligonucleotide as a primer to obtain an amplification product, the third oligonucleotide having a complementary sequence to a part or the whole of the nucleotide sequence of a sixth single-stranded nucleic acid or a seventh single-stranded nucleic acid, the sixth single-stranded nucleic acid including an oligonucleotide having a complementary sequence complementary to the first neighboring nucleotide sequence and the first oligonucleotide in this order from the 5′ side, the seventh single-stranded nucleic acid including an oligonucleotide having a complementary sequence to the first oligonucleotide and an oligonucleotide having the first neighboring nucleotide sequence in this order from the 5′ side as a primer. This method may further include a step of performing a nucleic acid amplification reaction using a fourth oligonucleotide and a fifth oligonucleotide as primers and using the amplification product as a template to obtain an amplification product, the fourth oligonucleotide having a complementary sequence to a part or the whole of the nucleotide sequence of the sixth single-stranded nucleic acid or the seventh single-stranded nucleic acid, and the fifth oligonucleotide including a sixth single-stranded nucleic acid having a second neighboring nucleotide sequence residing 1 to 1000 nucleotides apart from the target base on the opposite side to a binding sequence of the fourth oligonucleotide across the target base on the amplification product.

### Advantageous Effects of Invention

The present invention made it possible to provide a circular single-stranded nucleic acid, a method for preparing the same, and a method for using the same.

### BRIEF DESCRIPTION OF DRAWINGS

FIG. 1 A diagram showing a step of extension from an index primer in an embodiment of the present invention.

FIG. 2 A diagram showing a step of extension from a first neighboring primer and a step of converting a double strand to a single strand in an embodiment of the present invention.

FIG. 3 A diagram showing an extension step when an index primer is immobilized to a solid-phase substrate in advance in an embodiment of the present invention.

FIG. 4 A diagram showing a step of circularizing a single-stranded nucleic acid, a rolling circle amplification (RCA) step, and a nucleic acid amplification reaction step in an embodiment of the present invention.

FIG. 5 A schematic diagram showing a system for analyzing sequence data obtained, in an embodiment of the present invention.

FIG. 6 represents a graph showing the number of copies of an amplification product obtained by amplifying a target base using an H1975 cell line in an embodiment of the present invention.

### DESCRIPTION OF EMBODIMENTS

One embodiment of the present invention is a circular single-stranded nucleic acid for determining a target base on a genomic DNA, which includes a first single-stranded nucleic acid which has the target base or its complementary base and is a part of one of the strands of the genomic DNA, and a second single-stranded nucleic acid which has an index sequence to serve as an index of a cell, from which the genomic DNA is derived. Hereinafter, embodiments of the present invention including a method for preparing and using the circular single-stranded nucleic acid will be described in detail with reference to Examples.

Unless otherwise specifically stated in embodiments and Examples, methods described in standard protocols such as M. R. Green & J. Sambrook (Ed.), Molecular cloning, a laboratory manual (4th edition), Cold Spring Harbor Press, Cold Spring Harbor, N.Y. (2012); F. M. Ausubel, R. Brent, R. E. Kingston, D. D. Moore, J. G. Seidman, J. A. Smith, K. Struhl (Ed.), and Current Protocols in Molecular Biology, John Wiley & Sons Ltd., or their modified or altered methods are used. Further, in the case where commercially available reagents, kits, or measurement devices are used, protocols attached thereto are used unless otherwise specifically stated.

It should be noted that the objects, features, advantages, and ideas of the present invention are apparent to those skilled in the art from the description of this specification, and those skilled in the art can easily reproduce the present invention from the description of this specification. The mode, the specific examples and the like described below represent preferred embodiments of the present invention, and are given for the purpose of illustration or explanation, and the present invention is not limited thereto. It is obvious to those skilled in the art that various alterations and modifications can be made according to the description of this specification without departing from the spirit and scope of the present invention disclosed in this specification.

### 1. Index Primer

In this embodiment, a single-stranded nucleic acid which has an index sequence to serve as an index of a cell or its complementary sequence constitutes an index primer unique to each cell. The index primer is preferably constituted by DNA, but is not limited thereto, and may include, for example, RNA or artificial nucleic acids. The number of bases of the index primer is not particularly limited, but is preferably 4 to 30 bases. For example, when the index primer is used for discriminating between cell populations, constitution of the index sequence by only one base of A, C, G, and T enables discrimination of 4 different types of populations, constitution by two bases selected from A, C, G, and T enables discrimination of 4²=16 types, and constitution by N bases enables discrimination of 4^{N} different types of populations.

It is preferred that the index primer has a single-stranded nucleic acid having a random nucleotide sequence at the 3′ side of the single-stranded nucleic acid having an index sequence or its complementary sequence. This single-stranded nucleic acid itself has a random sequence, and in addition, each of the single-stranded nucleic acids has randomly diverse sequences. The number of bases of the random sequence is not particularly limited, but is preferably a number by which the sequence is capable of forming a complementary strand stably binding to the nucleic acid, and is, for example, preferably from 2 to 20 bases, more preferably from 4 to 10 bases.

The index primer may have a single-stranded nucleic acid having an adaptor sequence capable of being used in the subsequent amplification step or sequencing step at the 3′ side or the 5′ side of a single-stranded nucleic acid having an index sequence or its complementary sequence, but preferably the adaptor reside at the 5′ side. A plurality of the adaptor sequences may be provided.

### 2. Target Base on Genomic DNA

The target base on a genomic DNA is the base whose kind is to be determined, and is not limited to one base, and may be a plurality of bases or may be a nucleotide sequence. The number of the sequence is also not particularly limited, and may be several tens of bases to several hundreds of bases.

Examples of the target base include bases which can have mutations. The type of mutation is not particularly limited, and examples of the mutation include a point mutation such as an SNP (single nucleotide polymorphism), an insertion mutation, a deletion mutation, and a substitution mutation. Further, a gene or a part of a gene may be the target base.

### 3. Target Site-Specific First Neighboring Primer

The single-stranded nucleic acid having a first neighboring nucleotide sequence residing 1 to 1000 bases apart from the target base constitutes a specific first neighboring primer specific for the target site. This neighboring primer may be constituted by DNA, but is not limited thereto, and may include, for example, RNA or artificial nucleic acids. The distance from the target base to the first neighboring nucleotide sequence is 1 to 1000 bases, but is preferably 10 bases to 600 bases, and more preferably 30 bases to 200 bases. This distance depends on the number of the bases capable of being amplified at a time and capable of being determined at a time. The number of bases of the first neighboring nucleotide sequence is not particularly limited, but is preferably from 10 to 40 bases, and more preferably from 15 to 30 bases.

### 4. Method for Preparing Circular Single-Stranded Nucleic Acid

The method for preparing a circular single-stranded nucleic acid, which is one embodiment of the present invention, includes a step of performing a nucleic acid amplification reaction using an index primer and a first neighboring primer specific for the target site as primers and using a genomic DNA as a template to obtain an amplification product, and a step of connecting both ends of one of the single-stranded nucleic acids of the obtained amplification product to circularize the nucleic acid. An extension reaction of the index primer and an extension reaction of the first neighboring primer may be performed once for each and may be performed a plurality of times like PCR. When the reactions are performed only once for each, the reaction with one primer may be performed, followed by adding the other primer and performing the reaction with the other primer, or the reaction may be performed after both of the primers are added simultaneously. Hereinafter, a detailed description will be given.

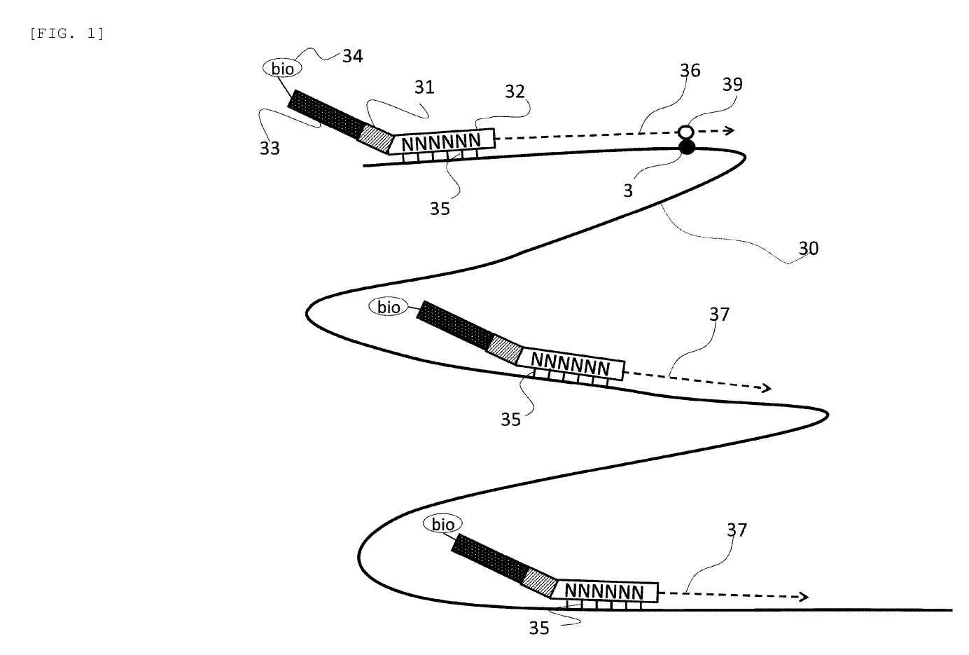

In a nucleic acid amplification reaction, first, random sequences in the index primer are hybridized to various places on the genome without bias, as shown in FIG. 1. Thereafter, an extension reaction of the index primer is performed. An enzyme having a strand displacement activity to extend an DNA strand while forcing another DNA strand having already complementarily bound ahead of the extended region to dissociate during the extension reaction of DNA is preferred. Examples of such an enzyme include Phi29 DNA polymerase, Bst DNA polymerase, Large Fragment, Deep vent DNA polymerase, and Klenow DNA polymerase. The DNA strand obtained by the extension has the index sequence at the 5′ side.

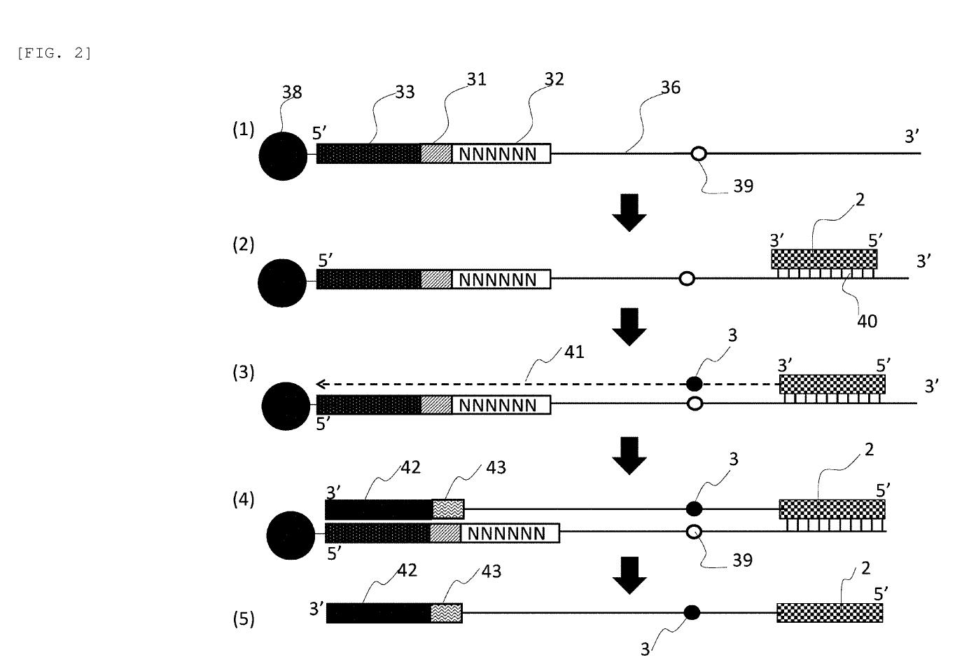

Subsequently, the first neighboring primer is hybridized to the obtained extended strand, as shown in FIG. 2. When the extended strand has the first neighboring nucleotide sequence, the first neighboring primer is hybridized to the extended strand. Thereafter, an extension reaction of the first neighboring primer is performed. An enzyme to be used here is not limited as long as it generally synthesizes a DNA strand, and may be a non-heat-resistant enzyme such as T4 DNA polymerase or Klenow DNA polymerase, or may be a heat-resistant enzyme such as Taq DNA polymerase, but is preferably a heat-resistant enzyme.

As described above, a double-stranded DNA having the index sequence at one end and the first neighboring nucleotide sequence at the other end is obtained.

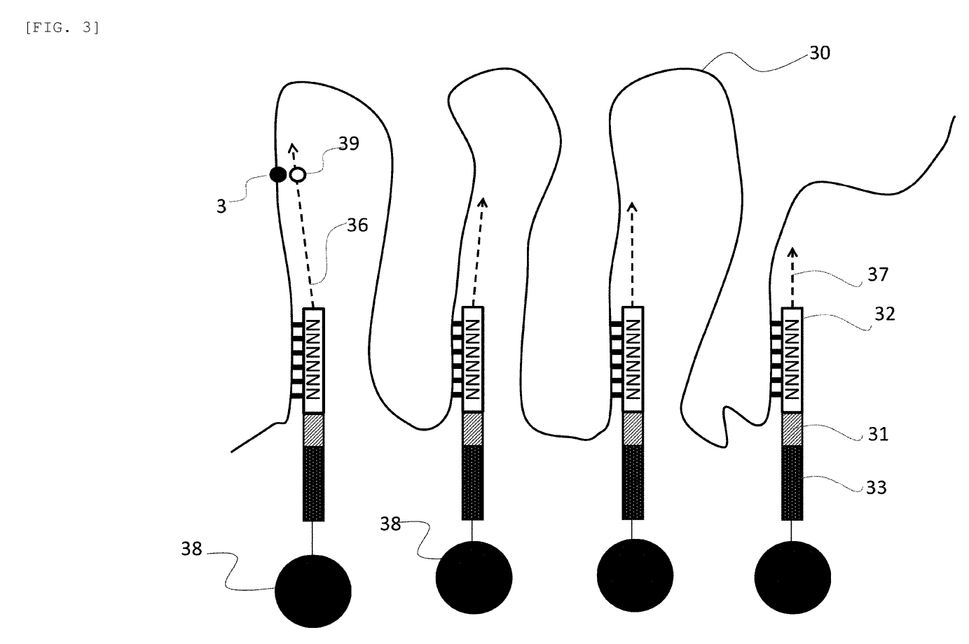

Subsequently, either one of the single-stranded DNAs is isolated from the double-stranded DNA. The method therefor is not particularly limited, but, for example, may include adding a substrate for isolation in advance to the 5′ end of the index primer or the first neighboring primer and be isolating it by utilizing a binding substance which binds to the substrate for isolation after the extension reaction as shown in FIG. 3. Examples of the substrate for isolation and the binding substance include a magnetic particle and a magnet, a biotin or its analogs and an avidin or its analogs, an antigen and an antibody, a His-tag and a metal, and GST and glutathione.

The substrate for isolation and the binding substance may be bound to each other before the extension step. By doing this, the reaction solution is easily exchanged. Further, in the case of a small amount of a sample, the loss due to adsorption onto a chip, a tube, or the like can be reduced.

Further, the binding substance may be bound to a solid-phase substrate. The solid-phase substrate is not particularly limited as long as it is made of a material which is generally used in the field of a nucleic acid analysis system for DNA and RNA. Examples thereof include metals such as gold, silver, copper, aluminum, tungsten, molybdenum, chromium, platinum, titanium, and nickel, and alloys such as stainless steel; silicon; glass materials such as glass, quartz glass, fused quartz, synthetic quartz, alumina, and photosensitive glass; plastics such as a polyester resin, polystyrene, a polyethylene resin, a polypropylene resin, an ABS resin (Acrylonitrile Butadiene Styrene resin), nylon, an acrylic resin, and a vinyl chloride resin; agarose, acrylamide, dextran, cellulose, polyvinyl alcohol, nitrocellulose, chitin, and chitosan.

The shape of the solid-phase substrate is not limited either, and may be a flat plate or the like, or may be a spherical bead, or the like, but is preferably a bead such as magnetic particles because it has a large binding surface as well as high operability.

When genomic DNA derived from a plurality of types of cells is present and there are a plurality of target bases in a sample, it is necessary to use an index primer having an index sequence different for each cell population. When each index primer is bound in advance to a solid-phase substrate, the solid-phase substrate may be made independent for each index primer by a known method.

As a method for converting a double-stranded DNA to a single strand, an enzymatic reaction can also be used. For example, the 5′ end of the index primer is modified in advance with a phosphate group, and one of the strands is degraded using an enzyme which specifically degrades a strand modified with the phosphate group at the 5′ end, whereby a double-stranded DNA can be converted to a single strand. Examples of the degrading enzyme include random exonuclease, although the enzyme is not limited as long as it shows a similar activity.

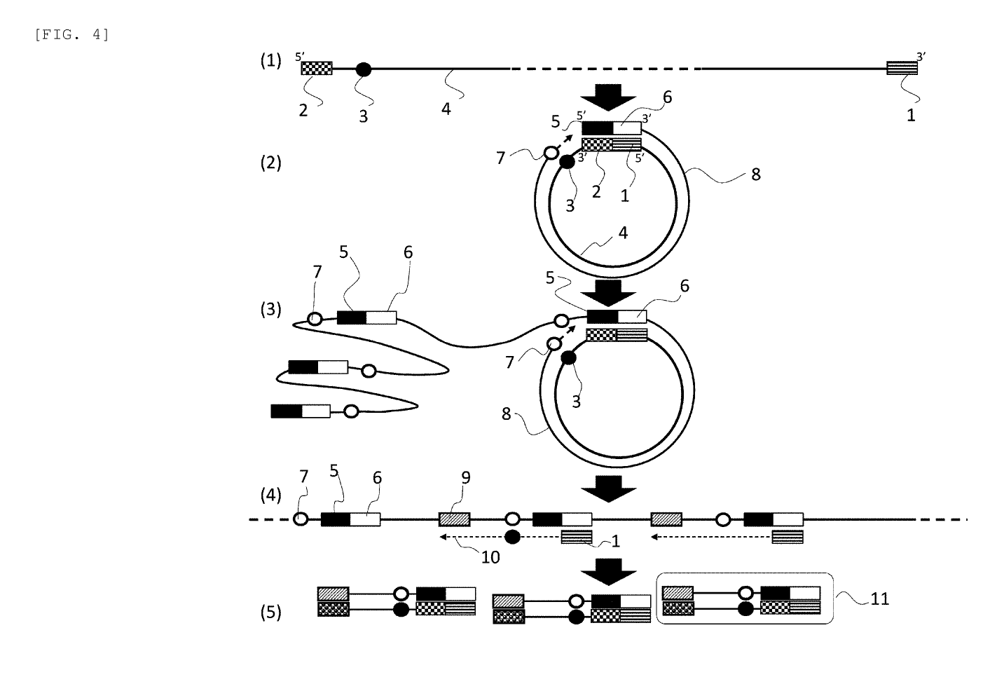

The both ends of either one of the single-stranded DNA thus obtained are connected to circularize the DNA. Enzymes such as Ampligase, T4 DNA ligase, Circligase, can be used for the circularization. In the case of Ampligase or T4 DNA ligase, a helper probe is preferably used for the circularization. The sequence of the helper probe is not particularly limited as long as it is an oligonucleotide which has complementary sequences to both terminal portions of the single-stranded DNA, and can hybridize thereto at the same time. For the single-stranded DNA in which the index primer is extended, an oligonucleotide having a nucleotide sequence in which a part or the whole of the 3′ terminal portion of the complementary nucleotide sequence to the index primer and a part or the whole of the 5′ terminal portion of the nucleotide sequence of the first neighboring primer are fused in this order from the 5′ side can be used as the helper probe to connect the 5′ end of the index primer and the 3′ end of the first neighboring primer. Specifically, when an adaptor sequence is not used, an oligonucleotide having a nucleotide sequence in which a part or the whole of the 3′ terminal portion of the complementary sequence to the index sequence and a part or the whole of the 5′ terminal portion of the nucleotide sequence of the first neighboring primer are fused in this order from the 5′ side can be used. When an adaptor sequence is used, an oligonucleotide having a nucleotide sequence in which a part or the whole of the 3′ terminal portion of the complementary sequence to the adaptor sequence and a part or the whole of the 5′ terminal portion of the nucleotide sequence of the first neighboring primer are fused in this order from the 5′ side or a nucleotide sequence in which a part or the whole of the 3′ terminal portion of the complementary sequence to the index sequence, the whole of the complementary sequence to the adaptor sequence, and a part or the whole of the 5′ terminal portion of the nucleotide sequence of the first neighboring primer are fused in this order from the 5′ side can be used. Further, for the single-stranded DNA generated by extension of the first neighboring primer, an oligonucleotide having a nucleotide sequence in which a part or the whole of the 3′ terminal portion of the complementary nucleotide sequence to the first neighboring primer and a part or the whole of the 5′ terminal portion of the nucleotide sequence of the index primer are fused in this order from the 5′ side can be used as the helper probe to connect the 3′ end of the index primer to the 5′ end of the first neighboring primer. Specifically, when an adaptor sequence is not used, an oligonucleotide having a nucleotide sequence in which a part or the whole of the 3′ terminal portion of the complementary sequence to the nucleotide sequence of the first neighboring primer and a part or the whole of the 5′ terminal portion of the index sequence are fused in this order from the 5′ side can be used. When an adaptor sequence is used, an oligonucleotide having a nucleotide sequence in which a part or the whole of the 3′ terminal portion of the complementary sequence to the nucleotide sequence of the first neighboring primer and a part or the whole of the 5′ terminal portion of the adaptor sequence are fused in this order from the 5′ side or a nucleotide sequence in which a part or the whole of the 3′ terminal portion of the complementary sequence to the nucleotide sequence of the first neighboring primer, the whole of the adaptor sequence, and a part or the whole of the 5′ terminal portion of the index sequence are fused in this order from the 5′ side can be used. It should be noted that, in FIG. 4, a step in the case where a single-stranded DNA 4 generated by extension of the first neighboring primer is used is shown.

Here, although the number of bases of the helper probe is not particularly limited, it is preferably from 10 to 100 bases, more preferably from 10 to 30 bases as a whole. And both of the numbers of bases on the side of the first neighboring primer and on the side of the index primer are preferably from 5 to 50 bases, more preferably from 5 to 15 bases, and preferably in particular, the numbers of the bases are the same on both sides.

### 5. Method for Using Circular Single-Stranded Nucleic Acid

Rolling circle amplification (RCA method) is performed using the circular single-stranded nucleic acid obtained as described above as a template, and using a complementary sequence to a part of the circularization product as a primer. An enzyme to be used in the RCA method is not limited as long as it has a strand displacement activity, and examples thereof include Phi29 DNA polymerase, Bst DNA polymerase, Vent DNA polymerase, and Deep Vent DNA polymerase. The primer to be used here is not particularly limited as long as it can be used to amplify the circular single-stranded nucleic acid, but it is preferred to use the helper probe used in the circularization for the sake of the easier processing. In this manner, as shown in FIG. 4(3), a single-stranded nucleic acid in which the nucleotide sequence of the helper probe and the nucleotide sequence of the genomic DNA are repeated is obtained.

Thereafter, a nucleic acid amplification reaction is performed for a nucleic acid around the target base in this single-stranded nucleic acid. As a specific nucleic acid amplification reaction, a known method such as Polymerase Chain Reaction (PCR method), Loop-Mediated Isothermal Amplification (LAMP method), Nucleic Acid Sequence-Based Amplification (NASBA method), or the like can be used. Primers to be used here are not particularly limited as long as they are a primer pair capable of amplifying the target base, but in the case of a single-stranded DNA derived from the circular single-stranded nucleic acid prepared with the single-stranded DNA generated by extension of the index primer, a first primer including a part or the whole of the 3′ terminal portion of the complementary sequence to the nucleotide sequence of the first neighboring primer and a second neighboring primer having the complementary sequence to a second neighboring nucleotide sequence residing 1 to 1000 bases apart from the target base on the opposite side to the first primer across the target base can be used. Similarly, in the case of a single-stranded DNA derived from the circular single-stranded nucleic acid prepared with the single-stranded DNA generated by extension of the first neighboring primer, a first primer including a part or the whole of the 3′ terminal portion of the nucleotide sequence of the first neighboring primer, and a second neighboring primer having the complementary sequence to a second neighboring nucleotide sequence residing 1 to 1000 bases apart from the target base on the opposite side to the first primer across the target base can be used.

Here, the distance from the target base to the second neighboring nucleotide sequence is 1 to 1000 bases, but is preferably 10 bases to 600 bases, and more preferably 30 bases to 200 bases. This distance depends on the number of nucleotides capable of being amplified at a time and capable of being determined at a time. The number of bases of the second neighboring nucleotide sequence is not particularly limited, but is preferably from 10 to 40 bases, more preferably from 15 to 30 bases.

The number of bases from the index primer to the target base varies, however, the number of bases from the first neighboring nucleotide sequence to the target base can be determined at discretion. Similarly, the number of bases from the second neighboring nucleotide sequence to the target base can be determined at discretion. Therefore, use of the above-mentioned primers enables determination of the number of bases suitable for the sequencing reaction to be performed after this step.

Further, when a plurality of target bases on the same genomic DNA is analyzed, the same index primer can be used, but the different sequences of the first neighboring primer and the second neighboring primer are required to be used for each target base. However, by setting the distances from each target base to these two neighboring primers to be the same or substantially the same, the constant efficiency of the sequencing reaction are enabled.

### 6. Use of Addition of Index to Target Base

Individual examination of a large number of cells enables the deepening of the statistical meaning of information in analyzing a genomic DNA or gene expression. Further, for example, also in the case of a plurality of types of small amounts of samples, more number of the types increases the accuracy of obtained information. On the other hand, when a large number of cells or samples are simultaneously analyzed, an index which is associated with the type of cell and indicates the origin of the cells or samples is useful. Even if genomes derived from a plurality of types of cells are mixed, it becomes possible with this index to discriminate which cell a genome is derived from.

In this manner, a target base can be analyzed while providing each of a plurality of types of cells having an index sequence specific to each type of cell and associating the index sequence including the target base. A detailed description will be given below.

(1) First, a case where the target base is the same and the kind of the base is different among cells is considered.

A plurality of types of cells are provided, and specific index primers having an index sequence having a sequence different for each cell type is used. As described above, double-stranded DNAs, which have an index sequence different for each cell type, and in which the first neighboring sequence and the second neighboring sequence are present across the target base, can be finally obtained.

Thereafter, when products obtained from all the cells are mixed and a sequencing reaction is performed as a whole, information of the index sequence and information of the target base can be obtained together. That is, the information of the target base becomes apparent separately for each cell.

(2) Subsequently, a case where the target base is present at different positions depending on the type of cells is considered.

A plurality of types of cells are prepared, and specific index primers having an index sequence having a sequence different for each type of the cell and different neighboring primers specific to each target base are used. As described above, double-stranded DNAs, which have an index sequence different for each cell type, and in which the first and the second neighboring sequences different for each cell type are present across the target base, can be finally obtained.

Thereafter, when products obtained from all the cells are mixed, and a sequencing reaction is performed as a whole, information of the index sequence and information of the target base can be obtained together. That is, the information of the target base becomes apparent for each cell.

### 7. Analysis of Obtained Sequence Data

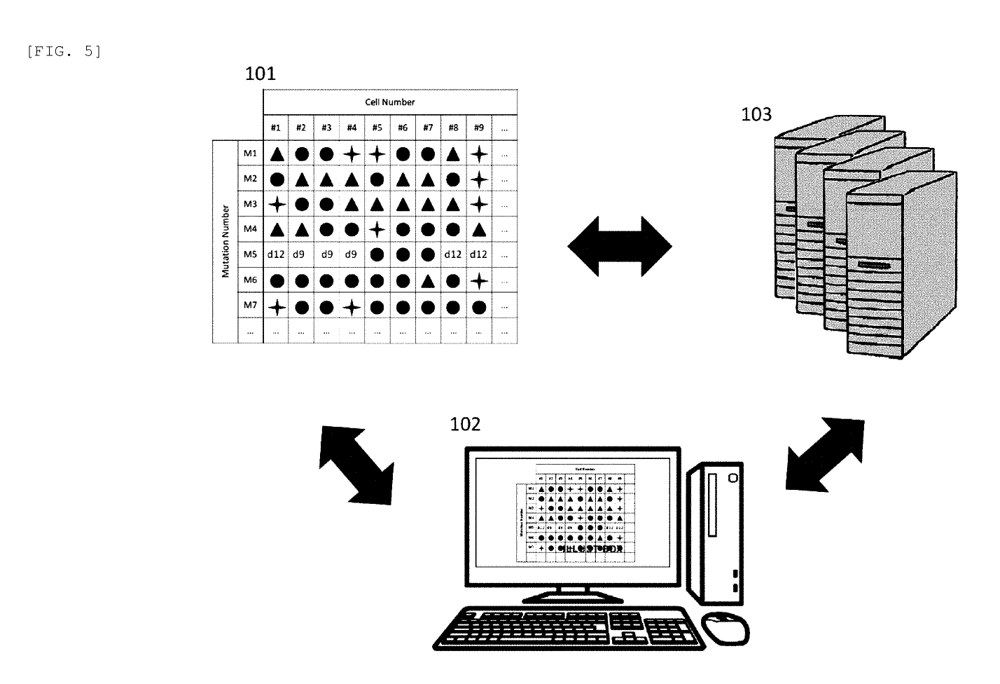

The sequence data of mutations derived from a large number of cells constructed by the above-mentioned method can be displayed as matrix data 101 organized according to the types of mutations and the cell numbers as shown in FIG. 5. By analyzing the combinations of mutation patterns by a computer for analysis 102, for example, it can be utilized as an index for administration of an anticancer agent for cancer, a disease caused by a mutation, or an index for a prognostic treatment. The types of mutations are numerous, and as the number of cells increases, the reliability of data increases. Therefore, it is possible to accumulate the organized data in a server 103, and to add a new finding thereto.

### EXAMPLES

Hereinafter, specific examples of embodiments of the present invention will be described. However, these examples are merely examples for embodying the present invention and do not limit the present invention.

In this Example, a point mutation at a target site to be analyzed in a genomic DNA extracted from a single cell was analyzed, using H1975 (cultured human pulmonary adenocarcinoma cells).

(1) First, as shown in FIG. 4, a step of obtaining a single-stranded DNA fragment having an index sequence and a target site-specific first neighboring primer (FIG. 4(1)), a step of making the target site to be analyzed and the index sequence adjacent to each other (FIG. 4(2)), and a step of amplifying the index sequence and the analysis target site next to each other (FIGS. 4(3 to 5)) will be described below.

As shown in FIG. 1, in this Example, an index primer 31 modified with a biotin label 34 at the 5′ end was used. As the index primer 31, an oligonucleotide constituted by the first adaptor sequence 33 (SEQ ID NO: 1) with a known sequence, a specific index sequence 31 (SEQ ID NOS: 2 to 11), and a 6-base random sequence 32 from the 5′ side was used. Their sequences are shown below.

(SEQ ID NO: 1)
5′-CGATGACGTAATACGACTCACTATAGGG-3′
(SEQ ID NO: 2)
5′-ATACGCG-3′
(SEQ ID NO: 3)
5′-GTACGCT-3′
(SEQ ID NO: 4)
5′-CGCTAGC-3′
(SEQ ID NO: 5)
5′-CTAGCGC-3′
(SEQ ID NO: 6)
5′-GTATCGC-3′
(SEQ ID NO: 7)
5′-CACGCTA-3′
(SEQ ID NO: 8)
5′-GATAGCG-3′
(SEQ ID NO: 9)
5′-CGAGCTA-3′
(SEQ ID NO: 10)
5′-CGCGACG-3′
(SEQ ID NO: 11)
5′-CGTCGCG-3′

In this Example, a plurality of index sequences 31 were attempted to be amplified. The adaptor sequence 33 is common to all index primers. N constituted by any nucleic acid of 4 types of A, C, G, and T was used in the random sequence 32. By constituting the index sequence 31 with 7 bases, 16,384 cells could be discriminated at the maximum.

The index primer 31 was fixed in advance to a streptavidin-labeled magnetic bead MyOne C1 (Thermo Scientific) (q=1 μm) 38 as a solid-phase substrate. It was fixed such that one copy of the primer and one magnetic bead were bound.

A genomic DNA extracted from a single cell, the index primer having a specific index, and 10⁷ beads to which the index primer was fixed were added to a reaction solution in each well of a 96-well plate. The detailed composition of the reaction solution is shown in Table 1. After the final volume of the reaction solution was adjusted to 10 μL with sterile water, an extension reaction was performed at 30° C. for 3 hours. Thereafter, the enzyme was inactivated by treatment at 60° C. for 10 minutes.

TABLE 1
Composition of Reagents forExtension Reaction

| Reagent | Reagent manufacturer | Final concentration | |
|---|---|---|---|
| Phi29 polymerase buffer | New England Biolabs | 1× | |
| dNTPs | Thermo Scienific | 2 | mM |
| BSA | | | |
| Phi29 DNA polymerase | New England Biolabs | 0.5 | u/μL |

(2) After completion of the extension reaction, the magnetic beads were washed with a buffer containing 10 mM Tris-Cl (pH 8.0) and 0.1% Tween using a neodymium magnet. After completely removing the supernatant, the magnetic beads were suspended in 2 μL of the same buffer. The random sequences 32 can form double strands 35 with the genomic DNA without bias, and extended strands 36 can be synthesized therefrom. Therefore, by this extension reaction, extended strands 36 which include and do not include the target base 39 in regions where the primers were extended are generated.

Subsequently, as shown in FIG. 2, a first neighboring primer 2 specific for the target site was annealed to the extended strand 36 of the index primer 31, and an extension reaction was performed. As the nucleotide sequence of the first neighboring primer 2, a sequence 5′-CCTGGCATGAACATGACCC-3′ (SEQ ID NO: 12) which is located on the opposite side to the index primer 31 across the target base 39 in the complementary strand 36 to the genomic DNA in which the target base is present and is 170 bases apart from the target base 39 was used. Therefore, the neighboring primer 2 annealed only to the extended strand 36 including the target base 39 to form a double strand 40 (FIG. 2(2)), and an extended strand 41 was synthesized using the extended strand 36 including the target base 39 as a template (FIG. 2(3)). Subsequently, the obtained double strand was denatured into a single strand, and then, the DNA strand attached to the magnetic bead was removed, whereby a single strand having the first neighboring primer 2 at the 5′ end and having a complementary sequence 43 to the specific index sequence and a complementary sequence 42 to the first adaptor sequence at the 3′ end was obtained (FIG. 2(5)).

Hereinafter, specific reaction conditions for the extension reaction are shown. An extension reaction reagent shown in Table 2 was added to the suspension of the magnetic beads to which the extended strand 36 of the index primer 31 was bound, and the final volume of the reaction solution was adjusted to 20 μL with sterile water. Subsequently, a thermal cycling reaction shown in Table 3 was performed using a thermal cycler.

TABLE 2
Composition of Extension Reaction Reagent
(Reagents were all from Thermo Scienific)

| Reagent | Final concentration | |
|---|---|---|
| First neighboring primer | 0.3 | μM |
| 10× Platinum HF buffer | 1× | |
| 50 mM MgSO₄ | 2 | mM |
| 10 mM dNTP | 0.2 | mM |
| Platinum Taq HF | 0.05 | u/μL |

TABLE 3
Conditions for Thermal Cycling Reaction

| | |
|---|---|
| Temperature | Time |

| | | | |
|---|---|---|---|
| 94° C. | 30 | sec | |
| 94° C. | 15 | sec | 5 cycles |
| 60° C. | 30 | sec | |
| 68° C. | 15 | min | |

After completion of the thermal cycling reaction, 5 μL of 0.5 N NaOH was added to the reaction solution (20 μL), and a denaturation reaction of the extended strand was performed by maintaining the reaction solution at 37° C. for 10 minutes. Subsequently, the supernatant was recovered while holding the magnetic beads with a neodymium magnet. In this supernatant, the extended strand of the first neighboring primer, which had been single-stranded, was contained. Subsequently, a neutralization reaction of the solution was performed by adding 5 μL of 0.5 N HCl thereto.

(3) Subsequently, the 5′ end of the resulting single-stranded DNA was subjected to a phosphorylation reaction. A phosphorylation reaction reagent shown in Table 4 was prepared, and the final volume of the solution was adjusted to 20 μL with sterile water. The reaction was performed at 37° C. for 30 minutes, and thereafter, the enzyme was inactivated at 70° C. for 5 minutes.

TABLE 4
Composition of Phosphorylation Reaction Reagent
(Reagents were all from Epicentre)

| Reagent | Final concentration | |
|---|---|---|
| Single-stranded product | | |
| 10× T4 PNK buffer | 1× | |
| 10 mM ATP | 1 | mM |
| T4 polynucleotide kinase | 0.06 | u/μL |

(4) Thereafter, the phosphorylated 5′ end and the 3′ end of the single-stranded DNA including the target base 3 were ligated, whereby a circularization product was synthesized. A circularization reaction reagent shown in Table 5 was prepared, and the final volume of the solution was adjusted to 10 μL with sterile water, and then, the reaction was performed at 50° C. for 1 to 5 hours. As the helper probe used in the circularization, an oligonucleotide composed of a complementary sequence (SEQ ID NO: 13) to both terminal portions of the DNA fragment as shown in FIG. 4(2) was used. The sequence used is shown below.

(SEQ ID NO: 13)
5′-GGGTCATGTTCATGCCAGGCGATGACGTAATACGACTCACTATAG
GG-3′

TABLE 5
Composition of Circularization Reaction Reagent
(Reagents were all from Epicentre)

| Reagent | Final concentration | |
|---|---|---|
| Phosphorylation product | | |
| 10× Ampligase buffer | 1× | |
| Ampligase | 0.5 | u/μL |
| Helper probe | 10 | μM |

(5) Subsequently, an amplification reaction by rolling circle amplification (RCA) with a helper probe was performed using the resulting circularization product as a template. An RCA product 8 has a structure in which a sequence of a complementary strand to the circularization product is repeated in parallel from a starting point of the helper probe as shown in FIG. 4(3).

Specifically, an RCA reaction reagent shown in Table 6 was prepared, and the final volume of the solution was adjusted to 10 μL with sterile water, and then, the reaction was performed at 37° C. for 2 to 16 hours.

TABLE 6
Composition of RCA Reaction Reagent (Reagents
were all from New England Biolabs)

| Reagent | Final concentration | |
|---|---|---|
| Circularization product | | |
| 10× Phi29 buffer | 1× | |
| Ampligase | 0.5 | u/μL |
| Helper probe | 10 | μM |

(6) Subsequently, as shown in FIGS. 4(4) and (5), by using a primer having a sequence of a complementary strand to the index primer and a second neighboring primer 9 composed of a complementary sequence (SEQ ID NO: 14) to the second neighboring sequence specific for the target site, and also using the RCA product as a template, an amplification reaction by a PCR reaction was performed, whereby a DNA fragment 10 including the target base 3 was amplified. The sequence used is shown below.

(SEQ ID NO: 14)
5′-CATCCTCCCCTGCATGTGT-3′

Hereinafter, the reaction is shown in detail. A PCR reaction reagent shown in Table 7 was added to the RCA product, and the final volume of the solution was adjusted to 25 μL with sterile water. Subsequently, a thermal cycling reaction shown in Table 8 was performed with a thermal cycler.

TABLE 7
Composition of PCR Reaction Reagent (Reagents
were all from Thermo Scienific)

| Reagent | Final concentration | |
|---|---|---|
| RCA product | | |
| Primer having sequence of complementary | 0.2 | μM |
| strand to first neighboring primer | | |
| Second neighboring primer | 0.2 | μM |
| 10× Platinum HF buffer | 1× | |
| 50 mM MgSO₄ | 2 | mM |
| 10 mM dNTP | 0.2 | mM |
| Platinum Taq HF | 0.05 | u/μL |

TABLE 8
Conditions for Thermal Cycling Reaction

| Temperature | Time | |
|---|---|---|
| 94° C. | 30 sec | |
| 94° C. | 15 sec | 35 cycles |
| 60° C. | 30 sec | |
| 68° C. | 60 sec | |

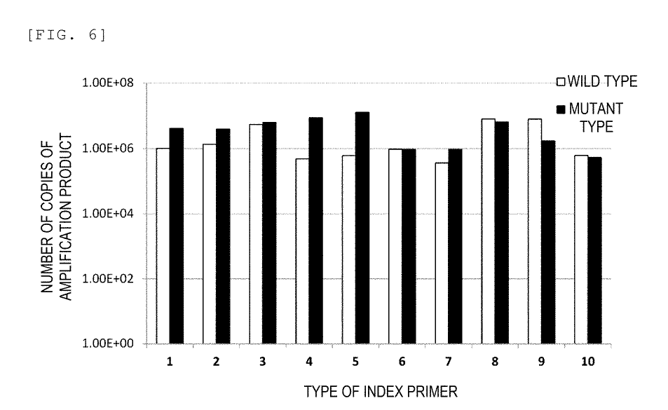

An amplification product of the DNA fragment in which the target base 3 and the specific index sequence 1 are adjacent to each other was thus obtained. The results of the quantitative determination of the amplification product are shown in FIG. 6.

The H1975 cell line is heterozygous for a mutation in the target base, and therefore, amplification products were obtained for both of the wild-type and the mutation. It should be noted that similar results were obtained also in the case of using any of the 10 types of index primers.

### REFERENCE SINGS LIST

1. index primer
2. first neighboring primer
3. target base
4. single-stranded DNA in which first neighboring primer is extended
5. sequence of complementary strand to first neighboring primer
6. sequence of complementary strand to index primer
7. complementary base to target base
8. RCA product
9. second neighboring primer
10. extension reaction of index primer
11. PCR product
30. genomic DNA
31. single-stranded nucleic acid having index sequence
32. single-stranded nucleic acid having random sequence
33. single-stranded nucleic acid having first adaptor sequence
34. biotin label
35. double strand
36. extended strand including target base
37. extended strand including no target base
38. magnetic bead
39. complementary base to target base
40. double strand
41. extended strand of first neighboring primer
42. single strand having complementary sequence to first adaptor sequence
43. single strand having index sequence
101. matrix data
102. PC for analysis
103. data server

1. A circular single-stranded nucleic acid for determining a target base on a genomic DNA, comprising:
a first single-stranded nucleic acid being a part of one of the strands of the genomic DNA and comprising the target base or a complementary base thereto; and
a second single-stranded nucleic acid comprising an index sequence to serve as an index of a cell or a complementary sequence thereto, the genomic DNA being derived from the cell.

2. The circular single-stranded nucleic acid according to claim 1, comprising a third single-stranded nucleic acid comprising an adaptor sequence or a complementary sequence thereto adjacent to the second single-stranded nucleic acid.

3. A method for preparing a circular single-stranded nucleic acid for determining a target base on a genomic DNA, comprising:
a step of performing a nucleic acid amplification reaction using a first oligonucleotide and a second oligonucleotide as primers and using the genomic DNA as a template to obtain an amplification product,
the first oligonucleotide comprising a second single-stranded nucleic acid having an index sequence to serve as an index of a cell, the genomic DNA being derived from the cell, and a fourth single-stranded nucleic acid having a random sequence in this order from the 5′ side,
the second oligonucleotide comprising a fifth single-stranded nucleic acid having a nucleotide sequence residing 1 to 1000 nucleotides apart from the target base or a complementary sequence thereto; and
a step of connecting both ends of one of the single-stranded nucleic acids of the amplification product to circularize the single-stranded nucleic acid.

4. The method for preparing a circular single-stranded nucleic acid according to claim 3, wherein the first oligonucleotide comprises, at the 5′ side of the second single-stranded nucleic acid, a third single-stranded nucleic acid having an adaptor sequence.

5. The method for preparing a circular single-stranded nucleic acid according to claim 3, wherein the fourth single-stranded nucleic acid is a population of single-stranded nucleic acids having random sequences.

6. The method for preparing a circular single-stranded nucleic acid according to claim 3, wherein the first oligonucleotide is bound to a solid-phase substrate.

7. A method for preparing a nucleic acid for determining a target base on a genomic DNA, comprising:
a step of performing a nucleic acid amplification reaction using a first oligonucleotide and a second oligonucleotide as primers and using the genomic DNA as a template to obtain an amplification product,
the first oligonucleotide comprising a second single-stranded nucleic acid having an index sequence as an index of a cell and a fourth single-stranded nucleic acid having a random sequence in this order from the 5′ side, the genomic DNA being derived from the cell, and
the second oligonucleotide comprising a fifth single-stranded nucleic acid having a first neighboring nucleotide sequence residing 1 to 1000 nucleotides apart from the target base or a complementary base thereto;
a step of connecting both ends of one of the single-stranded nucleic acids of the amplification product to circularize the single-stranded nucleic acid to obtain a circular single-stranded nucleic acid; and
a step of performing a rolling circle amplification (RCA) reaction using the circular single-stranded nucleic acid as a template and using a third oligonucleotide as a primer to obtain an amplification product, the third oligonucleotide comprising a complementary sequence to a part or the whole of the nucleotide sequence of a sixth single-stranded nucleic acid or a seventh single-stranded nucleic acid,
the sixth single-stranded nucleic acid comprising an oligonucleotide having a complementary sequence to the first neighboring nucleotide sequence and the first oligonucleotide in this order from the 5′ side,
the seventh single-stranded nucleic acid comprising an oligonucleotide having a complementary sequence to the first oligonucleotide and an oligonucleotide having the first neighboring nucleotide sequence in this order from the 5′ side.

8. The method according to claim 7, further comprising a step of performing a nucleic acid amplification reaction using a fourth oligonucleotide and a fifth oligonucleotide as primers and using the amplification product as a template to obtain an amplification product,
the fourth oligonucleotide having a complementary sequence to a part or the whole of the nucleotide sequence of the sixth single-stranded nucleic acid or the seventh single-stranded nucleic acid, and
the fifth oligonucleotide comprising a sixth single-stranded nucleic acid having a second neighboring nucleotide sequence residing 1 to 1000 nucleotides apart from the target base on the opposite side to a binding sequence of the fourth oligonucleotide across the target base on the amplification product.


SEQUENCE LISTING (14 sequences)

SEQ ID NO: 1 - DNA, 28 bp, Homo sapiens
cgatgacgtaatacgactcactataggg

SEQ ID NO: 2 - DNA, 7 bp, Artificial Sequence
  index seq
atacgcg

SEQ ID NO: 3 - DNA, 7 bp, Artificial Sequence
  index seq
gtacgct

SEQ ID NO: 4 - DNA, 7 bp, Artificial Sequence
  index seq
cgctagc

SEQ ID NO: 5 - DNA, 7 bp, Artificial Sequence
  index seq
ctagcgc

SEQ ID NO: 6 - DNA, 7 bp, Artificial Sequence
  index seq
gtatcgc

SEQ ID NO: 7 - DNA, 7 bp, Artificial Sequence
  index seq
cacgcta

SEQ ID NO: 8 - DNA, 7 bp, Artificial Sequence
  index seq
gatagcg

SEQ ID NO: 9 - DNA, 7 bp, Artificial Sequence
  index seq
cgagcta

SEQ ID NO: 10 - DNA, 7 bp, Artificial Sequence
  index seq
cgcgacg

SEQ ID NO: 11 - DNA, 7 bp, Artificial Sequence
  index seq
cgtcgcg

SEQ ID NO: 12 - DNA, 19 bp, Homo sapiens
cctggcatgaacatgaccc

SEQ ID NO: 13 - DNA, 47 bp, Artificial Sequence
  linker
gggtcatgttcatgccaggcgatgacgtaatacgactcactataggg

SEQ ID NO: 14 - DNA, 19 bp, Homo sapiens
catcctcccctgcatgtgt